(19) United States
(12) Patent Application Publication
(10) Pub. No.: US 2022/0143316 A1
(43) Pub. Date: May 12, 2022

(54) SYSTEM WITH A DEVICE AND A PROCESS FOR CONTROLLING AN ADMINISTRATION OF A DRUG TO A PATIENT

(71) Applicant: Drägerwerk AG & Co. KGaA, Lübeck (DE)

(72) Inventor: Hinrich ALTHOFF, Lübeck (DE)

(21) Appl. No.: 17/519,146

(22) Filed: Nov. 4, 2021

(30) Foreign Application Priority Data
Nov. 12, 2020 (DE) 10 2020 129 891.9

(51) Int. Cl. A61M 5/172

(52) U.S. Cl. CPC A61M 5/1723; A61M 2205/3576; A61M 2205/50; A61M 2205/18

(57) ABSTRACT

A device, a system including the device, to a process and to a computer program for controlling the administration of a drug to a patient. The device (10) comprises a processing device (11) configured to receive information concerning vital data of the patient (31), generation of a control signal in relation to a control parameter for controlling the administration of a drug with a drug administration unit (21) to the patient (31) on the basis of the information received concerning vital data of the patient. The device (10) comprises, furthermore, an interface (12), which is configured for providing the control signal for the drug administration unit (21).

### CROSS REFERENCE TO RELATED APPLICATIONS

This application claims the benefit of priority under 35 U.S.C. § 119 of German Application 10 2020 129 891.9, filed Nov. 12, 2020, the entire contents of which are incorporated herein by reference.

### TECHNICAL FIELD

The present invention pertains to a system comprising a device, a process and a computer program for controlling the administration of a drug to a patient, especially but not exclusively to a concept for the automated control and interruption of the administration of a drug to a patient.

### TECHNICAL BACKGROUND

Drugs may be administered in different dosage forms. For example, an administration of drugs with a drug administration (delivery) unit is possible. Many liquid drugs are administered with syringe pumps or infusion pumps in an intensive care unit. As a rule, pumps can be started and stopped exclusively manually. Certain drugs, for example, catecholamines, may strongly affect the vital data of the patient, for example, the blood pressure. The dosage rate of the drug is typically set manually such that the blood pressure will remain within a defined window. This happens possibly several times a day. At the same time, the vital data of the patient can be monitored continuously by patient monitoring by means of a patient monitor. When the value exceeds or drops below a limit value, the patient monitor can generate an alarm. Furthermore, a continuous measurement can be made possible with a urimeter. The excretion of urine can in this case be used as a vital parameter. Further, a continuous measurement of glucose in the blood may also be monitored as a vital parameter in connection with the administration of insulin.

TCI (Target Controlled Infusion) pumps are also employed for administering drugs. In case of TCI pumps for target-controlled infusion for anesthesia, the dosage rate of the pump can be controlled by software and be continuously adapted according to pharmacokinetic models. TCI can automate the dosage of intravenous drugs during surgery. After the desired parameters have been set on the device by the anesthesiologist and the start button has been pressed, the infusion pump controls the rate of delivery, while the administration is being monitored by the anesthesiologist. TCI can thus be just as safe and effective as manually controlled infusions. TCI pumps were developed originally for the dispensing of propofol. In this case the anesthesiologist only selects the desired target concentration of the drug in the plasma. The rate of delivery is then set by an algorithm in the pump and is set based on a time curve. The algorithm is based on a pharmacokinetic model, which images the time curve of the physiological processes from the intravenous administration of the drug to the concentration in the plasma. The models are comparatively simple and they typically include the manually entered body weight, height and gender of the patient, but not current measured values or vital data.

A scientific publication, “Target controlled infusion” (TCI)—A concept with a future? Determination of location, recommendations for action and view into the future,” S. Schraag, S. Kreuer, J. Bruhn, C. Frenkel, S. Albrecht; The Anaesthetist, March 2008, relates to TCI. Further details on using a syringe pump, for example, an instruction for use of a syringe pump are presented in the document “Alaris™ PK Syringe Pump Model 80053UN01 Directions For Use” that can be downloaded at: https://www.bd.com/documents/international/guides/directions-for-use/infusion/1000DF00331.pdf. The use of the syringe pump according to model 80053UN01 of the manufacturer is described there.

Drawbacks of the conventional pumps are that the dosage rate adjustment is based on statistical and static models. In addition, TCI pumps have been approved so far for use under continuous supervision by an anesthesiologist only. One problem is that the continuous administration of a drug by syringe pumps is started and stopped exclusively manually in intensive care units.

### SUMMARY

Against this background, there is therefore a need for the administration of a drug such that the drug administration is adapted individually to a patient/to the state of a patient. This need is met by a system and process according to the invention.

Exemplary embodiments are based on the core idea of controlling the administration of a drug to a patient with a drug administration unit on the basis of received information concerning vital data of the patient. A control parameter can in this case indicate whether and how a dosage rate for administering a drug can be increased, can be reduced or can be left unchanged or whether the administration of the drug can be interrupted. For example, the administration of a drug can be interrupted on the basis of critical vital data. Furthermore, an automatic dose adjustment or interruption of the administration of the drug can take place when the patient is measurably in a clinically critical range or shows a trend towards such a range.

Exemplary embodiments of the system according to the present invention create a device for controlling the administration of a drug to a patient. The device comprises an administration device configured for continuously receiving information concerning vital data of the patient. The administration device is further configured for generating a control signal in relation to a control parameter for controlling the administration of a drug with a drug administration unit to the patient on the basis of the information received concerning vital data of the patient. The device comprises, furthermore, an interface, which is configured to provide the control signal to the drug administration unit. The control parameter may in this case indicate for example, whether and how a dosage rate for administering the drug can be increased, can be reduced, can remain unchanged or that the administration of the drug is interrupted. This can make possible a simple and reliable control of the administration of the drug to the patient as a function of the vital data of the patient.

The information concerning vital data of the patient may in this case comprise an alarm signal that vital data of the patient are in a critical range. The interruption of the administration of the drug to the patient can be made subject to alarms in this case.

The alarm signal may in this case comprise a “high” alarm state, which indicates that vital data of the patient rise above an upper alarm limit, and/or a “low” alarm state, which indicates that vital data of the patient drop below a lower alarm limit. It can be determined in this manner whether the upper alarm limit was exceeded or the vital data dropped below the lower alarm limit.

The administration device may, furthermore, be configured, for example, to make available the control parameter to the drug administration unit on the basis of the information concerning vital data of the patient. The control parameter may in this case indicate whether and how a dosage rate for administering the drug can be increased, can be lowered, can be left unchanged or whether the administration of the drug can be interrupted. The dosage rate can be controlled reliably with the control parameter and it can be set individually for the patient.

Furthermore, the control signal generated by the administration device may comprise a plurality of control parameters. Each individual control parameter may be configured for controlling the administration of a drug, associated with the respective control parameter, to the patient. The administration of a plurality of drugs to the patient can be controlled in a reliable manner in this case.

The administration device may further be configured in some exemplary embodiments to generate the control parameter on the basis of the information received concerning vital data of the at least two different vital data of the patient. Different vital data of the patient can be included in this case in order to control the administration of the drug to the patient. The administration of the drug to the patient can be improved in this case even more and it can be adapted even more individually to the respective situation or to the state of the patient.

The administration device may, furthermore, be configured in additional exemplary embodiments to make available the control parameter as a set point in a control, wherein the information concerning vital data of the patient act as a measured value in the control and the control has an integral component. This can make possible a control with a high accuracy, especially also with a steady-state (stationary) accuracy.

The system according to the present invention comprises, in addition to the device, the drug administration unit. The administration of a drug can be controlled thereby continuously with the system in a simple and reliable manner.

The drug administration unit may in this case be configured in the form of a syringe pump and/or of an infusion pump. As a result, the drug can be administered to the patient reliably.

Furthermore, the system according to the present invention comprises a vital data measuring device. As a result, the functionality of the system can be expanded and the administration of the drug to the patient can be controlled continuously even more simply.

The vital data measuring device may be a patient monitor and/or a urimeter. The patient monitor and/or the urimeter can make it possible for the health care staff to detect the vital data of the patient and optionally additional data rapidly and clearly.

The drug administration unit is especially preferably configured for administering catecholamines and the vital data measuring device is especially preferably configured for measuring the blood pressure of the patient.

In some exemplary embodiments, the vital data measuring device may be configured to generate the alarm signal in relation to an alarm parameter, which indicates whether the information concerning vital data of the patient is below or above a predefined limit value. This can make it possible for the health care staff to detect rapidly and reliably whether the vital data of the patient are below or above the predefined limit value.

In additional exemplary embodiments, the device, the drug administration unit and/or the vital data measuring device may be configured to communicate by means of a shared communication protocol in a network. The communication of the device, of the drug administration unit and/or of the vital data measuring device can be made in this case more reliable and be further simplified.

Furthermore, exemplary embodiments create a process for controlling the administration of a drug to a patient. The process comprises continuous receipt of information concerning vital data of the patient and generation of a control signal in relation to a control parameter for controlling the administration of a drug with a drug administration unit to the patient on the basis of the information concerning vital data of the patient. The administration of the drug can in this case be controlled as a function of the vital data of the patient.

Another exemplary embodiment is a computer program with a program code for carrying out a process being described here when the program code is executed on a computer, on a processor or on a programmable hardware component. A machine-readable data storage medium with such a program code is an additional exemplary embodiment.

Some examples of devices and/or processes will be explained in more detail below only as examples with reference to the attached figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a block diagram of an exemplary embodiment of an administration device for controlling the administration of a drug to a patient;

FIG. 2 is a block diagram of an exemplary embodiment of a system comprising the administration device;

FIG. 3 is a block diagram of an exemplary embodiment of another system comprising the device; and

FIG. 4 is a block diagram of an exemplary embodiment of a process for controlling the administration of a drug to a patient.

### DESCRIPTION OF PREFERRED EMBODIMENTS

Referring to the drawings, different examples will be described now in more detail in reference to the attached figures. The thicknesses of lines, layers and/or areas may be exaggerated for illustration in the figures.

Further examples may cover modifications, equivalents and alternatives, which fall within the scope of the disclosure. Identical or similar reference numbers refer in the entire description of the figures to identical or similar elements, which may be implemented identically or in a modified form in a comparison with one another, while they provide the same function or a similar function.

It is apparent that if an element is described as being “connected” or “coupled” to another element, the elements may be connected or coupled directly or via one or more intermediate elements. When two elements A and B are combined with the use of an “or,” this shall be defined as that all possible combinations are disclosed, i.e., only A, only B as well A and B, unless this is explicitly or implicitly defined as being otherwise. An alternative wording for the same combinations is “at least one of A and B” or “A and/or B.” The same applies, mutatis mutandis, to combinations of more than two elements.

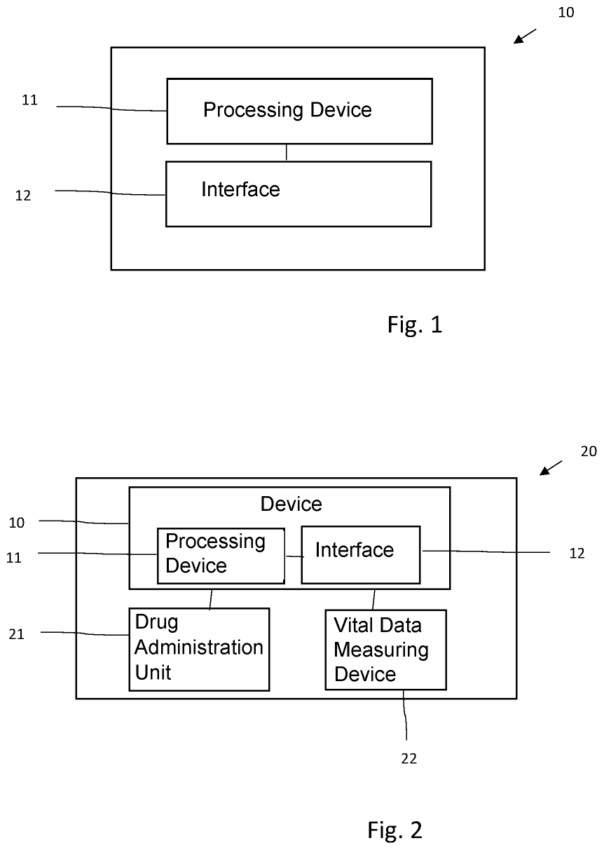

FIG. 1 shows a block diagram of an exemplary embodiment of a device (administration device) 10 for controlling the administration of a drug to a patient. The administration device 10 comprises a processing device 11 configured for receiving information concerning vital data of the patient. The processing device 11 is further configured to generate a control signal in relation to a control parameter for controlling the administration of a drug with a drug administration unit to a patient on the basis of information received concerning vital data of the patient. The processing device 11 may be configured in the form of a processor, especially in the form of a system-on-a-chip.

The processing device 11 may comprise in exemplary embodiments one or more freely selectable controllers, microcontrollers, network processors, processor cores, such as digital signal processor cores (DSPs), programmable hardware components, etc. Exemplary embodiments are not limited here to a defined type of processor core. Freely selectable processor cores or even a plurality of processor cores or microcontrollers may be provided for implementing a processing device 11. Implementations in an integrated form with other devices are also conceivable, for example, in a control unit, which additionally also comprises one or more other functions. A processing device 11 may be embodied in exemplary embodiments by a processor core, by a computer processor core (CPU=Central Processing Unit), by a graphics processor core (GPU=Graphics Processing Unit), by an application-specific integrated circuit (ASIC=Application-Specific Integrated Circuit), by an integrated circuit (IC=Integrated Circuit), by a one-chip system core (SOC=System on Chip), by a programmable logic element or by a field-programmable gate array with a microprocessor (FPGA=Field Programmable Gate Array) as a core of the component or of the components.

Examples of vital data of the patient may be a respiratory rate, a blood pressure, an oxygen saturation and/or a heart rate of the patient. The control parameter may indicate, for example, whether and how a dosage rate for administering a drug is increased, is decreased, is left unchanged or the administration of the drug is interrupted. A window, in which the vital parameter shall be, can be predefined or set by a user, for example, based on an instruction given by a physician. The window may be defined by an upper alarm limit and by a lower alarm limit. The user may be a physician and/or health care staff The device and/or a system may comprise for this purpose a user interface. The user interface may be configured, for example, as a display, a touchscreen, a keyboard and/or a mouse. The drug administration unit may be configured, for example, in the form of a syringe pump and/or of an infusion pump. A syringe pump is defined as a metering pump for the continuous parenteral administration of drugs. An infusion pump, also called volumetric pump, is defined as a metering pump for the continuous, predominantly intravenous administration of infusions. Further, other drug administration units may be used as well.

The administration device 10 comprises, furthermore, an interface 12, which is configured for providing the control signal for the drug administration unit and which is coupled with the processing device 11. The interface 12 may be configured, for example, in the form of a machine interface or in the form of a software interface.

The interface 12 may be configured in exemplary embodiments as a typical interface for communication in networks or between network components or medical devices. For example, this interface 12 may be configured in exemplary embodiments by corresponding contacts. It may also be configured in exemplary embodiments as separate hardware and comprise a memory, which stores the signals to be transmitted or to be received at least temporarily. The interface 12 may be configured for receiving electrical signals, for example, as a bus interface, as an optical interface, as an Ethernet interface, as a wireless interface, as a field bus interface, etc. It may, moreover, be configured in exemplary embodiments for wireless transmission and comprise a radio front end as well as corresponding antennas.

The information concerning vital data of the patient may comprise an alarm signal that vital data of the patient are in a critical range. The alarm signal may comprise, for example, three states, a “high” alarm state showing that vital data of the patient are too high (alarm state “high”) and rise above an upper alarm limit; a “low” alarm state showing that vital data of the patient are too low (“low” alarm state) and drop below a lower alarm limit, and a normal state. The alarm state may indicate, for example, that vital data of the patient are in a clinically critical range, either above or below a clinically noncritical window, while the normal state may indicate, for example, that the vital data of the patient are not in a clinically critical range. The alarm signal may comprise, in addition, an acoustic signal, which is outputted. For example, the administration of a blood pressure-lowering drug can be interrupted in this manner only when the measured blood pressure drops below the lower alarm limit, but it is not interrupted when the blood pressure rises above the upper alarm limit.

Furthermore, the processing unit 11 is configured to make available the control parameter for the drug administration unit on the basis of the information concerning vital data of the patient. The vital data of the patient and/or variables derived from them, which go beyond the three states, namely, the “high” alarm state, the “low” alarm state and the normal state, can be transmitted now to the processing unit 11 as information concerning vital data of the patient. The control parameter can indicate now whether and how a dosage rate for administering the drug is increased, decreased, left unchanged or the administration of the drug is interrupted.

Further, the control signal generated by the processing unit 11 may comprise a plurality of control parameters. Each individual control parameter may in this case be configured for controlling the administration of a drug associated with the respective control parameter to the patient. The administration of a plurality of drugs to the patient can be controlled in this case. Each control parameter can in this case indicate in reference to the drug associated with it, for example, whether and how a dosage rate for administering the drug is increased, decreased, left unchanged or the administration of the drug is interrupted.

For example, a vital value of a patient is outside of a predefined range (e.g., blood pressure is too low) in a simple exemplary embodiment. Corresponding information, e.g., an indicator indicating this relationship, or a bit/flag, is then received. The administration of a blood pressure-lowering drug is in this case interrupted, which is indicated to the drug administration unit by the processing device 11 by means of the control signal. The control signal may then likewise comprise a simple indicator, a bit, a flag or an interrupt signal (interruption signal).

The processing device 11 may be configured, furthermore, to generate the control parameter on the basis of the received information concerning vital data of at least two different vital parameters of the patient. Different vital data, for example, respiratory rate, blood pressure, oxygen saturation and/or heart rate may in this case be included in the generation of the control parameter for the administration of a drug.

Furthermore, the processing device 11 may be configured to provide the control parameter as a set point in a control. The information concerning vital data of the patient may in this case act as a measured value in the control and the control may have an integral (integration) component. The integral component may have a stationary (steady-state) accuracy. Furthermore, the control may have a proportional component and/or a differential component.

The exemplary embodiment shown in FIG. 1 may comprise one or more additional optional features.

FIG. 2 shows a block diagram of an exemplary embodiment of a system 20 comprising the administration device 10. The system 20 comprises the above-described administration device 10 and the drug administration unit 21. The drug administration unit 21 may be configured in the form of a syringe pump and/or of an infusion pump. A syringe pump is defined as a metering pump for the continuous parenteral administration of drugs. An infusion pump, also called a volumetric pump, is defined as a metering pump for the continuous, predominantly intravenous administration of infusions. Further, other drug administration units may be used as well.

The system 20 may comprise, furthermore, a vital data measuring device 22. The vital data measuring device 22 may be a patient monitor. Patient monitors may be used to measure and monitor vital data of the patient. Patient monitors may be used, for example, during the anesthesia during surgery, in critically ill patients or in other clinical situations, which require continuous monitoring. The patient monitor may also be configured as a mobile device for use with emergency patients.

The vital data measuring device 22 may be configured in this connection to generate the alarm signal in relation to an alarm parameter, which indicates whether the information concerning vital data of the patient is below or above a predefined limit value. The drug administration unit 21 or pump shut-off may use a separate limit value, which differs from the limit value or from the alarm limit value of the vital data measuring device 22 or of the patient monitor. The properties concerning the alarm generation may remain, for example, unchanged from conventional methods, for example, in the case of the patient monitor.

Further, the administration device 10, the drug administration unit 21 and/or the vital data measuring device 22 may be configured to communicate in a network by means of a shared communication protocol. For example, IEEE 11073 SDC (Service-oriented Device Connectivity) may also be used as a shared communication protocol. Other communication protocols may be used as well.

The IEEE (Institute of Electrical and Electronics Engineers) 11073 SDC standard family defines a communication protocol for medical devices, whose field of use is in the potentially critical patient care, e.g., in the operating room, in the intensive care unit, but in other areas of the hospital as well. It is part of the ISO/IEEE 11073 standard family. IEEE 11073 SDC is based on the paradigm of the service-oriented architecture, SOA. The introduction of SDC as a standard for the bidirectional communication of measured values and set points between medical devices may also open up possibilities of remote control. Closed-loop systems especially for controlling the administration of drugs with syringe pumps may be complicated concerning their validation, because large studies may be necessary. A useful intermediate step may be represented by automatic emergency shut-offs, which can help avoid critical situations.

The exemplary embodiment shown in FIG. 2 may comprise one or more additional optional features.

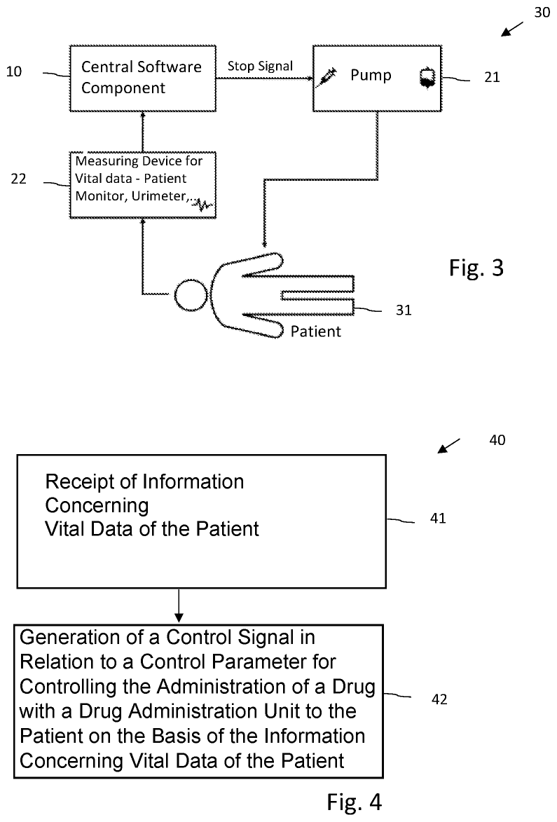

FIG. 3 shows a block diagram of an exemplary embodiment of another system 30 comprising the administration device 10. The system 30 may be implemented similarly to system 20, which is described in conjunction with FIG. 2. The system 30 comprises the administration device 10, the drug administration unit 21 and the vital data measuring device 22. The administration device 10 is in this case configured as a central software component, which is executed on corresponding hardware. The administration device 10 is implemented as is described in connection with FIG. 1, and the administration device comprises, among other things, the processing device 11 described there and the interface 12 as hardware components. The administration device 10 is connected to the vital data measuring device 22. The vital data measuring device 22 or measuring device for vital data is configured for measuring vital data of the patient 31. Further, the vital data measuring device 22 may also be configured for the especially simultaneous measurement of different vital data, for example, heart rate, respiratory rate, oxygen saturation and/or blood pressure of the patient 31.

The drug administration unit may be configured for administering catecholamines in this exemplary embodiment or in one of the other exemplary embodiments mentioned. The vital data measuring device is preferably configured for simultaneously measuring the blood pressure of the patient. As a result, the system according to the present invention can advantageously be used for a catecholamine therapy.

The vital data measuring device 22 may be configured, for example, as a patient monitor and/or urimeter. The urimeter may be configured as a sterile, closed collection system with integrated measuring chamber. The urimeter may comprise, furthermore, a vertical drainage system and built-in nonreturn valves, which ensure with certainty that the measuring chamber can be completely emptied after a measurement in order to make it possible to guarantee accurate measurements especially in case of low urine output. The urimeter, which may be configured similarly to a hydrometer, may be used, for example, for measuring a specific gravity or a flow of a urine. The specific gravity of the urine may be a function of the number, the density and the weight of particles dissolved in the urine and can be used as an indicator of the kidney's ability to concentrate urine.

The vital data measuring device 22 may be connected to the patient 31 for measuring the vital data of the patient 31. For example, the vital data measuring device 22 may comprise a cuff, which may be configured to be placed around an arm of the patient 31 and thus to be able to determine the blood pressure of the patient 31. Furthermore, an invasive, arterial blood pressure measurement may be carried out. The invasive blood pressure measurement, IBP, is a form of blood pressure measurement in which a sensor is inserted directly into an artery of the patient via an arterial access. The artery may be punctured with a cannula and be connected to a liquid-filled tube system. A pressure change within the artery can be passed on in the liquid-filled tube system to a pressure transducer, also called transducer. A pressure wave can be recorded there by a membrane and be converted into electrical pulses, which can be displayed by a connected measuring device. Furthermore, the vital data measuring device 22 may comprise a heart rate measuring device, for example, in the form of a chest belt and/or skin electrodes in order thus to be able to determine the heart rate of the patient 31. The vital data measuring device 22 can transmit information concerning the vital data of the patient 31, for example, via an air interface or a cable connection to the administration device 10. The administration device 10 generates a control signal in relation to a control parameter for controlling the administration of a drug with the drug administration unit 21 to the patient 31 on the basis of the information concerning vital data of the patient 31.

The administration device 10 in this case can generate a stop signal as a control signal when information concerning vital data of the patient 31 indicates that the vital data of the patient 31 are in a clinically critical range. The stop signal may be, for example, a high alarm state, when vital data of the patient are too high (“high” alarm state), and a “low” alarm state, when vital data of the patient are too low (“low” alarm state). The alarm state may indicate, for example, that vital data of the patient are in a clinically critical range, either above or below a clinically noncritical window. The administration device 10 can transmit in this case the stop signal to the drug administration unit 21 in order to interrupt the administration of the drug to the patient 31 with the drug administration unit 21.

For example, the administration of a drug with a syringe pump can be interrupted when a vital parameter influenced by the drug exceeds or drops below a set limit value. For example, a central unit in the network, such as the administration device 10, can monitor for this purpose the alarms of a patient monitor. At the same time, the unit can have information on which syringe pumps with which drugs are connected to the same patient 31. As soon as the corresponding alarm is present at the device or vital data measuring device 22 and it is communicated into the network, for example, by means of SDC, the unit can send a remote control command to the corresponding syringe pump or drug administration unit 21 in order to interrupt the administration. In a situation that is critical for the patient 31, it is then possible not only to generate an alarm, but also to automatically interrupt a possibly causal or at least contraindicated drug administration. A prerequisite for this may be that pumps shall allow the possibility of remote control in order to interrupt an administration.

According to one exemplary embodiment, the patient 31 in the intensive care unit can receive dobutamine in order to treat an excessively low blood pressure of the patient 31. The alarm limits of the patient monitor can in this case be set in a relatively narrow range in order to detect a further drop in the blood pressure despite the medication early on, on the one hand, and, on the other hand, in order to interrupt the administration of the drug when the blood pressure rises into a range critical for the patient 31. Should the blood pressure rise now based on pathophysiological circumstances, e.g., above the alarm limit of 140 mmHg mean pressure, the patient monitor generates an alarm correspondingly and communicates the alarm into the network. The central unit receives the alarm there and sends the command to stop the feed to the drug administration unit 21 or to a dobutamine pump. A dobutamine pump can be used to dispense dobutamine to the patient 31.

The exemplary embodiment shown in FIG. 3 may comprise one or more additional optional features.

FIG. 4 shows a block diagram of an exemplary embodiment of a process 40 for controlling the administration of a drug to a patient 31. The process 40 comprises the receipt 41 of information concerning vital data of the patient. The process 40 comprises, furthermore, the generation 42 of a control signal in relation to a control parameter for controlling the administration of a drug with a drug administration unit to the patient 31 on the basis of the information concerning vital data of the patient 31. The control parameter may in this case indicate whether and how a dosage rate for administering the drug is increased, reduced, left unchanged or the administration of the drug is interrupted. The dosage rate of the drug administration unit 21 or of a pump can in this case be controlled by the administration device 10. The dosage rate can optionally be lowered to a preset value or it can be reduced to zero. In one application, the central unit or administration device 10 can detect on the basis of rules that the dosage rate is too high or too low in relation to the vital data of the patient 31 and interrupt the administration of the drug in order to prevent an incorrect medication based on the current state of the patient.

According to another exemplary embodiment, the blood pressure of the patient can be controlled with a drug. A drug stop, with which the administration of the drug with the drug administration unit 21 is stopped, can in this case be triggered based on the heart rate of the patient. A patient 31 with sepsis can receive phenylephrine as a vasopressor via the drug administration unit 21 or a central venous catheter in order to treat the excessively low blood pressure. The central venous catheter may be configured as a thin plastic tube, which is inserted into a venous system via a vein of an upper half of the body and whose end may be located in an upper or lower vena cava in front of the right atrium of the heart. Substances that can be used to raise or to support the blood pressure can be called vasopressors. The consequences are an increased vasotonia, increased contractility of the heart and increasing cardiac pumping volume.

The drug may, however, also affect the heart rate and trigger bradycardia, in addition to affecting the blood pressure. Bradycardia is a slowed-down heartbeat and is used in medicine to describe a heart rate below 60 beats per minute in adult humans. When the heart rate of the patient 31 drops below the set limit value of, e.g., 40 per minute, the administration by the pump or by the drug administration unit 21 can be stopped.

In another exemplary embodiment, the patient 31 can be administered liquid at a rate of 150 mL/hour via an infusion pump or the drug administration unit 21 into a central access or into the central venous catheter. The infusion pump is a metering pump for the continuous, predominantly intravenous administration of infusions. When the central venous pressure becomes too high, the infusion pump can be stopped or the rate of the pump or infusion pump can be reduced, for example, to 50 mL/hour, cf. also the exemplary embodiment concerning the treatment of the patient 31 with adrenaline.

According to another exemplary embodiment, the patient 31 can receive the active ingredient furosemide in order to enhance the diuresis. Furosemide is a drug from the group of the loop diuretics. Loop diuretics lead to the excretion of large quantities of tissue fluid by inhibiting a transport protein in the kidney. Diuresis is called the urine excretion by the kidneys. A urimeter can measure the excretion and the fact that a defined quantity or a rate of excretion at which the furosemide administration shall be interrupted is reached.

Exemplary embodiments may, furthermore, be or pertain to a computer program with a program code for executing or carrying out one or more of the above processes when the computer program is executed on a computer, on a processor or on a programmable hardware component or on the processing device 11. Steps, operations or procedures of different processes described above can be carried out by programmed computers or processors. Examples may also cover program memory devices, e.g., digital memory media, which are machine-, processor- or computer-readable and code machine-executable, processor-executable or computer-executable programs of instructions. The instructions carry out some or all of the steps of the above-described processes or cause them to be executed. The program memory devices may comprise or be, e.g., digital memories (flash memories or solid state drive memories), magnetic storage media, for example, magnetic disks and magnetic tapes, hard drives or optically readable digital storage media. Further examples may also cover computers, processors or control units, which are programmed to execute the steps of the above-described processes, or (field)-programmable logic arrays ((F)PLAs (Field) Programmable Logic Arrays) or (field)-programmable gate arrays ((F)PGA=(Field) Programmable Gate Arrays), which are programmed to execute the steps of the above-described processes.

The exemplary embodiment shown in FIG. 4 may comprise one or more additional optional features.

Functions of different elements shown in the figures as well as the designated function blocks may be implemented in the form of dedicated hardware, e.g., of “a signal provider,” of “a signal processing unit,” of “a processor,” of “a control”, etc., as well as hardware capable of executing software in conjunction with corresponding software. In case of provision by a processor, the functions may be provided by an individual dedicated processor, by an individual, jointly used processor or by a plurality of individual processors, some of which or all of which may be used jointly. However, the term “processor” or “control” is far from being limited to hardware capable exclusively of executing software, but it may comprise digital signal processor hardware (DSP hardware; DSP=Digital Signal Processor), network processor, application-specific integrated circuit (ASIC=Application Specific Integrated Circuit), Field Programmable Gate Array (FPGA), read-only memory (ROM=Read Only Memory) for storing software, random-access memory (RAM=Random Access Memory) and non-volatile storage device. Other hardware, conventional and/or customer-specific, may be included as well.

A block diagram may represent, for example, a schematic circuit diagram, which implements the basic principles of the disclosure. Similarly, a flow chart, a flow diagram, a state transition diagram, a pseudocode and the like may represent different processes, operations or steps, which are represented, for example, essentially in computer-readable medium and are thus executed by a computer or processor, regardless of whether such a computer or processor is explicitly shown. Processes disclosed in the specification or in the patent claims may be implemented by a component that has a means for executing each of the respective steps of these processes.

It is apparent that the disclosure of a plurality of steps, processes, operations or functions disclosed in the specification or in the claims shall not be considered to be in the defined order, unless this is explicitly or implicitly stated otherwise, e.g., for technical reasons. Therefore, these are not limited to a defined order by the disclosure of a plurality of steps or functions, unless these steps or functions are not replaceable for technical reasons. Further, an individual step, function, process or operation may have in some examples a plurality of partial steps, partial functions, partial processes or partial operations and/or be broken up into these. Such partial steps may be included and be a part of the disclosure of this individual step, unless they are explicitly excluded.

While specific embodiments of the invention have been shown and described in detail to illustrate the application of the principles of the invention, it will be understood that the invention may be embodied otherwise without departing from such principles.

### LIST OF REFERENCE NUMBERS

10 Administration device
11 Processing device
12 Interface
20 System
21 Drug administration unit
22 Vital data measuring device
30 System
31 Patient
40 Process
41 Receipt of information concerning vital data of the patient
42 Generation of a control signal in relation to a control parameter for controlling the administration of a drug

What is claimed is:

1. A system comprising:
a vital measuring device configured to continuously monitor vital data of a patient;
a drug administration unit operatively connected to the patient and configured to administer a drug to the patient based on a control signal;
an administration device configured to control an administration of the drug to the patient, the administration device comprising:
a processing device configured to continuously receive information concerning vital data of the patient from the vital data measuring device, to generate the control signal in relation to a control parameter for controlling the administration of the drug to the patient with the drug administration unit based on the information received concerning vital data of the patient; and
an interface operatively connected to the drug administration unit and configured to provide the control signal for the drug administration unit.

2. A system in accordance with claim 1, wherein the information concerning vital data of the patient comprises an alarm signal that vital data of the patient are in a critical range.

3. A system in accordance with claim 2, wherein the alarm signal comprises:
a high alarm signal, which indicates that vital data of the patient has risen above an upper alarm limit; or
a low alarm signal, which indicates that vital data of the patient has dropped below a lower alarm limit; or
a high alarm signal, which indicates that vital data of the patient has risen above an upper alarm limit and a low alarm signal, which indicates that vital data of the patient has dropped below a lower alarm limit.

4. A system in accordance with claim 1, wherein the control parameter indicates whether a dosage rate for administering the drug is to be increased, reduced, left unchanged or if the administration of the drug is to be interrupted and indicates how the dosage rate for administering the drug is to be increased, reduced, left unchanged or interrupted.

5. A system in accordance with claim 1, wherein the control signal generated by the processing device comprises a plurality of control parameters, wherein each individual control parameter is configured for controlling the administration of a drug associated with the respective control parameter to the patient.

6. A system in accordance with claim 1, wherein the processing device is further configured to generate the control parameter on the basis of the information received concerning vital data of at least two different vital parameters of the patient.

7. A system in accordance with claim 1, wherein:
the processing device is configured to provide the control parameter as a set point in a control; and
the information concerning vital data of the patient act as a measured value in the control and the control has an integral component.

8. A system in accordance with claim 1, wherein the drug administration unit comprises one or more of a syringe pump and an infusion pump.

9. A system in accordance with claim 1, wherein:
the drug administration unit is configured for administering catecholamines; and
the vital data measuring device is configured for the measurement of blood pressure of the patient.

10. A system in accordance with claim 2, wherein the vital data measuring device is configured for generating the alarm signal in relation to an alarm parameter, which indicates whether the information concerning vital data of the patient is below or above a predefined limit value.

11. A system in accordance with claim 1, wherein the administration device, the drug administration unit and/or the vital data measuring device are configured to communicate by means of a shared communication protocol in a network.

12. A process for controlling the administration of a drug to a patient, the process comprising the steps of:
providing a system comprising: a vital measuring device configured to continuously monitor vital data of a patient; a drug administration unit operatively connected to the patient and configured to administer a drug to the patient based on a control signal; and an administration device configured to control an administration of a drug to a patient, the administration device comprising: a processing device configured to continuously receive information concerning vital data of the patient from the vital data measuring device, to generate the control signal in relation to a control parameter for controlling the administration of the drug with the drug administration unit to the patient based on the information received concerning vital data of the patient; and an interface operatively connected to the drug administration unit and configured to provide the control signal for the drug administration unit;
continuous receiving the information concerning vital data of the patient at the administration device from the vital measuring device; and
generating the control signal, with the processing device, in relation to the control parameter for controlling the administration of a drug with the drug administration unit to the patient based on the information concerning vital data of the patient.

13. A process in accordance with claim 12, further comprising providing a program code for carrying out at least one of the process steps upon the program code is executed.

14. A process in accordance with claim 12, wherein the information concerning vital data of the patient comprises an alarm signal that vital data of the patient are in a critical range.

15. A process in accordance with claim 14, wherein the alarm signal comprises:
a high alarm signal, which indicates that vital data of the patient has risen above an upper alarm limit; or
a low alarm signal, which indicates that vital data of the patient has dropped below a lower alarm limit; or
a high alarm signal, which indicates that vital data of the patient has risen above an upper alarm limit and a low alarm signal, which indicates that vital data of the patient has dropped below a lower alarm limit.

16. A process in accordance with claim 12, wherein the control parameter indicates whether a dosage rate for administering the drug is to be increased, reduced, left unchanged or if the administration of the drug is to be interrupted and indicates how the dosage rate for administering the drug is to be increased, reduced, left unchanged or interrupted.

17. A process in accordance with claim 12, wherein the control signal generated by the processing device comprises a plurality of control parameters, wherein each individual control parameter is configured for controlling the administration of a drug associated with the respective control parameter to the patient.

18. A process in accordance with claim 12, wherein the processing device is further configured to generate the control parameter on the basis of the information received concerning vital data of at least two different vital parameters of the patient.

19. A process in accordance with claim 12, wherein:
the processing device is configured to provide the control parameter as a set point in a control;
the information concerning vital data of the patient act as a measured value in the control and the control has an integral component.

20. A process in accordance with claim 12, wherein the drug administration unit comprises one or more of a syringe pump and an infusion pump.
